(19) United States
(12) Patent Application Publication
(10) Pub. No.: US 2024/0008847 A1
(43) Pub. Date: Jan. 11, 2024

(54) ULTRASOUND FOR NEURO-IMAGING AND NEURO-MODULATION DEVICE IN A SINGLE DEVICE

(71) Applicant: X Development LLC, Mountain View, CA (US)

(72) Inventors: Joel Emilio Bregman Segre, Oakland, CA (US); Vladimir Miskovic, Binghamton, NY (US)

(21) Appl. No.: 17/858,143

(22) Filed: Jul. 6, 2022

(51) Int. Cl. A61B 8/00; A61N 7/00; A61B 8/08; A61B 8/13; G01S 15/89

(52) U.S. Cl. CPC A61B 8/4488; A61N 7/00; A61B 8/0808; A61B 8/54; A61B 8/13; G01S 15/8915; A61N 2007/0026; A61N 2007/0052

(57) ABSTRACT

A system includes: an ultrasound array comprising a plurality of transducer elements, wherein: a first subset of the plurality of transducer elements are configured to emit ultrasound pulses through the subject's skull for performing a neuro-modulation of the subject's brain, and a second subset of the plurality of transducer elements are configured to receive ultrasound signals from the subject's skull and brain in response to the ultrasound pulses being emitted from the first subset of the plurality of transducer elements; and a controller coupled to the ultrasound array, wherein the controller is configured to: generate at least one image depicting at least a portion of the subject's skull and brain based on, at least in part, the ultrasound signals received by the second subset of the plurality of transducer elements, and adapt the neuro-modulation of the subject's brain based on, at least in part, the at least one image.

### FIELD

This specification relates to transcranial brain stimulation and brain imaging.

### BACKGROUND

Stimulation of the brain in humans is typically performed using electrical or magnetic fields without feedback and with respect to a generic position relative to a subject's head, and typically is not based on measurements of the particular subject's brain activity or tailored to the particular subject's brain morphology or cranial structure. Moreover, brain imaging is typically performed using computer tomography (CT) or magnetic resonance imaging (MRI).

### SUMMARY

In one aspect, some implementations provide a system for imaging and neuro-modulation of a subject's brain through the subject's skull, the system comprising: an ultrasound array comprising a plurality of transducer elements, wherein: a first subset of the plurality of transducer elements are configured to emit ultrasound pulses through the subject's skull for performing a neuro-modulation of the subject's brain during use of the system, and a second subset of the plurality of transducer elements are configured to receive ultrasound signals from the subject's skull and brain in response to the ultrasound pulses being emitted from the first subset of the plurality of transducer elements; and a controller coupled to the ultrasound array, wherein the controller is configured, during use of the system, to: generate at least one image depicting at least a portion of the subject's skull and brain based on, at least in part, the ultrasound signals received by the second subset of the plurality of transducer elements, and adapt the neuro-modulation of the subject's brain based on, at least in part, the at least one image.

Implementations may include one of more of the following features.

The controller may be further configured, during use of the system, to: determine at least one characteristic of the subject's skull based on, at least in part, the at least one image, and adjust at least one parameter for emitting the ultrasound pulses from the first subset of the plurality of transducer elements to adapt to the at least one characteristic of the subject's skull. The controller may be further configured, during use of the system, to store a plurality of templates, wherein the plurality of templates comprise data encoding images of known skulls, and wherein the images are generated based on ultrasound signals received from the known skulls in response to the ultrasound pulses being directed to the known skulls from the first subset of the plurality of transducer elements.

The controller may be further configured, during use of the system, to train skull models based on, at least in part, the plurality of templates and the at least one image specific to the subject; and wherein each skull model comprises at least one feature that corresponds to a skull thickness. When the at least one parameter is adjusted, the ultrasound pulses being emitted from the first subset of the plurality of transducer elements may be adapted to the skull thickness specific to the subject.

The first subset of the plurality of transducer elements and the second subset of the plurality of transducer elements may share a common group of transducer elements. Each of the second subset of the plurality of transducer elements may be sized to be smaller than at least one transducer element from the first subset of the plurality of transducer elements. The first and the second subset of the plurality of transducer elements may be tuned to operate between 200 kHz to 2 MHz. The at least one image comprises a tomographic image of the subject's brain. The first subset of the plurality of transducer elements may be configured, during use of the system, to surround the subject's skull.

In another aspect, implementations may provide a method for imaging and neuro-modulation of a subject's brain through the subject's skull, the method comprising: emitting, by a first subset of a plurality of transducer elements, ultrasound pulses through the subject's skull for performing a neuro-modulation of the subject's brain; receiving, by a second subset of the plurality of transducer elements, ultrasound signals reflected from the subject's brain and skull in response to the ultrasound pulses being emitted from the first subset of the plurality of transducer elements; generating at least one image depicting at least a portion of the subject's skull and brain based on, at least in part, the ultrasound signals received by the second subset of the plurality of transducer elements; and adapting the neuro-modulation of the subject's brain based on, at least in part, the at least one image. The method may further include: determining at least one characteristic of the subject's skull based on, at least in part, the at least one image, and adjusting at least one parameter for emitting the ultrasound pulses from the first subset of the plurality of transducer elements to adapt to the at least one characteristic of the subject's skull. The method may further include: accessing a plurality of templates that comprise data encoding images of known skulls, wherein the images are generated based on ultrasound signals received from the known skulls in response to the ultrasound pulses being directed to the known skulls from the first subset of the plurality of transducer elements.

The method may further include: building skull models based on, at least in part, the plurality of templates and the at least one image specific to the subject, wherein each skull model comprises at least one feature that corresponds to a skull thickness. When the at least one parameter is adjusted, the ultrasound pulses being emitted from the first subset of the plurality of transducer elements may be adapted to the skull thickness specific to the subject.

The first subset of the plurality of transducer elements and the second subset of the plurality of transducer elements may share a common group of transducer elements. Each of the second subset of the plurality of transducer elements may be sized to be smaller than at least one transducer element from the first subset of the plurality of transducer elements. The first and the second subset of the plurality of transducer elements may be tuned to operate between 200 kHz to 2 MHz. The at least one image may include a tomographic image of the subject's brain. The method may further comprise: arranging the first subset of the plurality of transducer elements to surround the subject's skull.

Other embodiments of this aspect include corresponding systems, apparatus, and computer programs, configured to perform the actions of the methods, encoded on computer storage devices.

The details of one or more implementations are set forth in the accompanying drawings and the description, below. Other potential features and advantages of the disclosure will be apparent from the description and drawings, and from the claims.

### BRIEF DESCRIPTION OF THE DRAWINGS

FIG. 1A is a diagram of an example configuration of a combined transcranial brain imaging and neuro-modulation system.

FIG. 1B is another diagram of an example configuration of a combined transcranial brain imaging and neuro-modulation system.

FIG. 2 is a diagram of an example of applying machine learning to a combined transcranial brain imaging and neuro-modulation system.

FIGS. 3A, 3B, 3C, 3D, 3E, and 3F are illustrations of example form factors of a combined transcranial brain imaging and neuro-modulation system.

FIG. 4 is a flow chart of an example process for combining transcranial imaging and neuro-modulation.

Like reference numbers and designations in the various drawings indicate like elements. The components shown here, their connections and relationships, and their functions, are meant to be examples only, and are not meant to limit the implementations described and/or claimed in this document.

### DETAILED DESCRIPTION

This technology relates to a device for both imaging and neuro-modulation using ultrasound. Current practice relies on other modalities such as MRI or CT for imaging and guidance while applying neuro-modulation. When ultrasound is used for neuro-modulation, the treatment frequently operates by assuming skull characteristics (and their resultant effects on ultrasound intracranial intensity) are identical in all users, even though the assumption is generally known to be empirically false. Both the reliance on other modalities and the assumption of skull thickness are prone to complications. The proposed device can use a set of ultrasound transducers for neuro-modulation of a subject's brain, as well as imaging (e.g., tomographic imaging) of the subject's skull and brain during, prior to, and/or after therapy. The set of ultrasound transducers may be arranged to surround the subject's skull. The neuro-stimulation may launch ultrasound waves from the set of transducers through the skull to form an intracranial focus where neuron activities are modulated by ultrasound insonification. For imaging, full wave inversion can be used to reconstruct the underlying anatomy of the brain and the skull.

Using the same set of ultrasound transducers for both imaging and stimulation has a number of advantages. Imaging can reveal anatomical information such as skull thickness and fatty layer for improved focusing of neuro-modulation, as well as individualized dosimetry. Imaging can also trace the actual path of the beam through the subject's skull and verify the accuracy in reaching an intended target of interest. In other words, imaging may use the same waveform as the one used for neuro-modulation. In some cases, a subset of the ultrasound transducers can be used to launch the ultrasound waveform for neuro-modulation while a different subset of the ultrasound transducers can be arranged to receive ultrasound signals. Each element of the subset of ultrasound transducers for receiving can be smaller than the element of the transmitting subset. In some examples, the transducers may operate at a center frequency of around 500 kHz with a fractional bandwidth of around 100%.

Notably, stimulation of particular regions of a brain, including large-scale brain networks—various sets of synchronized brain areas linked together by brain function—can be used to treat neurological and psychiatric disorders and certain effects of physical disorders. The methods and systems described here can be used for therapeutic purposes to treat psychiatric conditions such as anxiety disorders, trauma and stressor-related disorders, panic disorders, and mood disorders as well as treating the physical symptoms of various disorders, diseases, and conditions. For example, the described system can be used to treat phobias, reduce anxiety, and/or control tremors or tinnitus, among other applications. Additionally, these methods can be used for cognitive remediation (e.g., improve or restore executive control), to improve alertness, and/or to aid sleep regulation, among other applications. These methods can also be used to produce positive effects on a subject's memory, attention, and focus. For example, the described method can be used to produce a desired psychological state in a subject, to aid in meditation, to increase focus, and/or to enhance learning and skill acquisition, among other applications.

Generally speaking, currently available brain stimulation methods are not personalized for particular subjects and their needs, and do not take into account skull structure or brain activity that occurs in response to the stimulation. These methods typically are not tailored to a particular subject's brain morphology or activity and such stimulation waveforms are often highly artificial (e.g., a square wave or random noise), without resembling natural patterns of brain activity.

In contrast, the described methods and systems perform transcranial stimulation of the brain, allow for stimulation of large-scale brain networks in real-time, and adjust the stimulation parameters, including frequency, power, focal length, time duration, and spot size, based on measurements taken of the subject's brain structure and activity patterns and cranial structure and the surrounding tissue, hair, and other biomaterial. These measurements can be used with statistical and/or machine learning models to determine a current brain state, to analyze the subject's response to the stimulation, and to determine future stimulation parameters. In some implementations, the measurements can be used to map out cranial and brain structure, connectivity, and functionality to personalize stimulation to a particular subject. For example, the described methods can include providing ultrasonic stimulation according to a particular set of stimulation parameters to a particular area of a subject's brain, contemporaneously or near-contemporaneously recording brain activity detected by sensors, adjusting stimulation parameters based on the detected brain activity, and applying the adjusted stimulation parameters.

The described methods and systems can be implemented automatically (e.g., without direct human control). For example, the controller can automatically detect and identify activity of a particular subject's brain and use the activity to tailor stimulation parameters and detection techniques to the particular subject's brain.

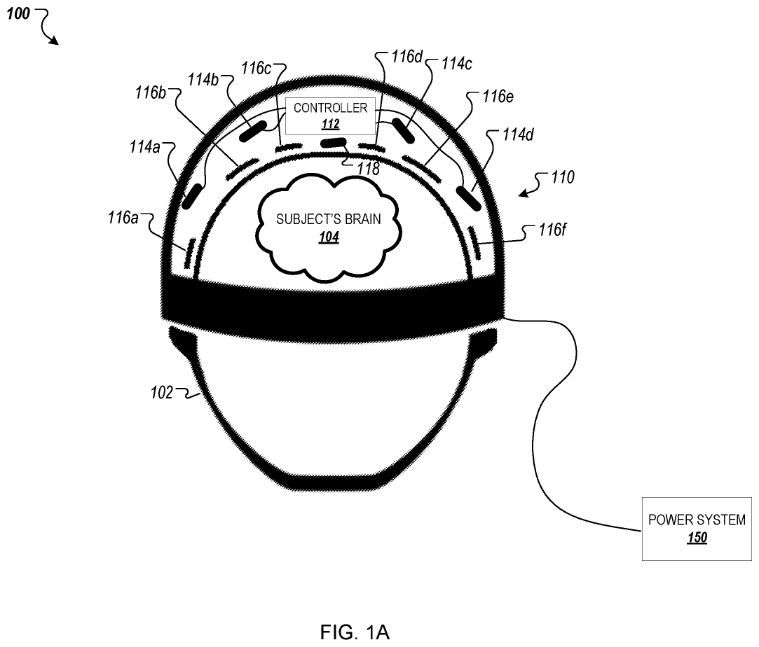

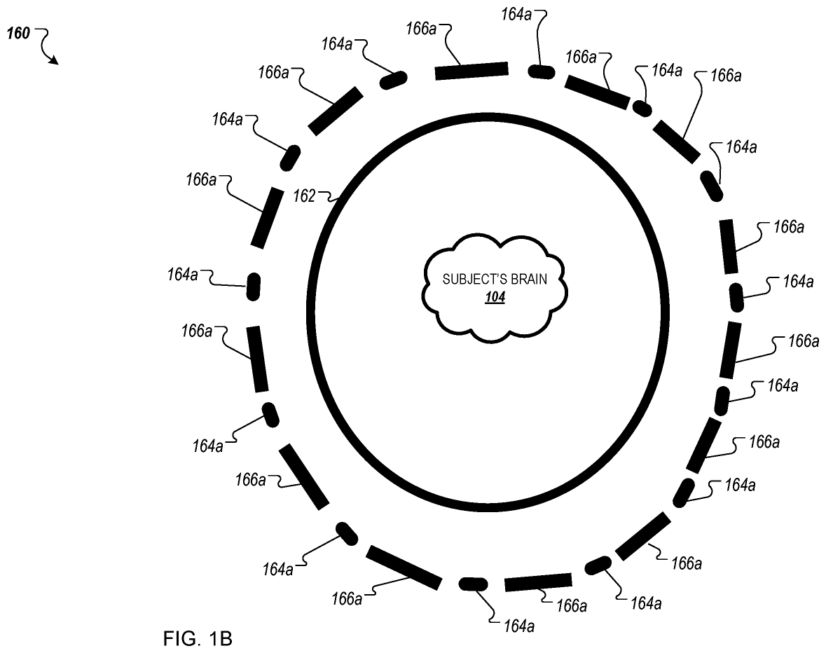

FIG. 1A is a diagram of an example configuration 100 of a transcranial brain imaging and neuro-modulation system 110. System 110 provides transcranial neuro-modulation as well as transcranial imaging of the subject's brain, both of which are premised on ultrasound. For example, system 110 can be used to stimulate a target area inside a subject's brain. Based on skull thickness information and brain anatomy map, as revealed by transcranial imaging, the system 110 can adjust various parameters for stimulating the target area.

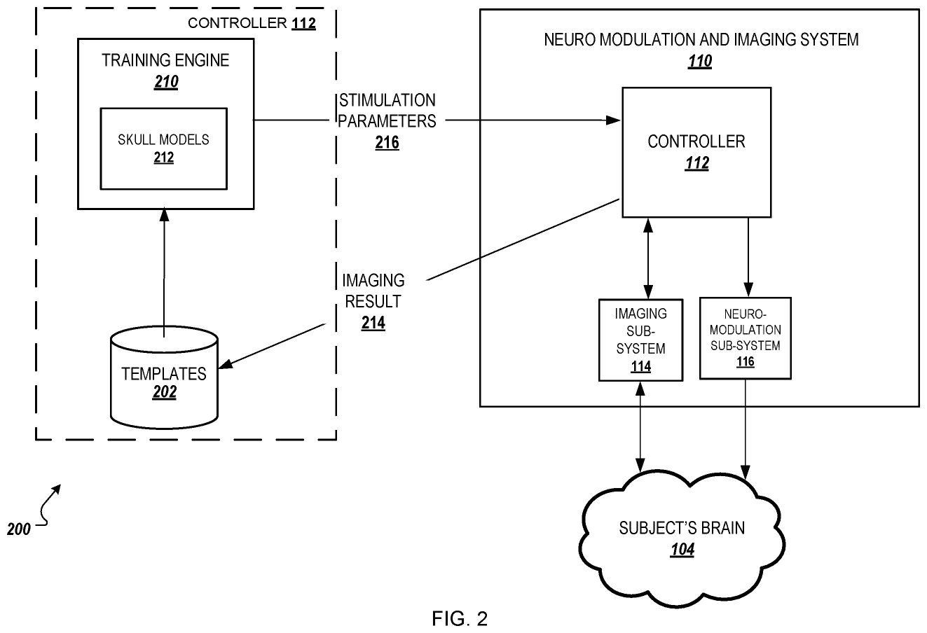

Further referring to FIG. 1B and FIG. 2, system 110 incorporates a transcranial ultrasonic neuro-modulation and imaging system that includes an imaging sub-system 114 and a neuro-modulation sub-system 116. Both the imaging sub-system 114 and neuro-modulation sub-system 116 have a first subset of ultrasound transducer elements (e.g., transducer elements 116a, 116b, 116c, 116d, 116e, and 116f) coupled to the subject's brain 104 through the skull so that the first subset of ultrasound transducer elements are configured, before and/or during use, to generate and direct a first ultrasound beam at a region within a portion of a subject's brain. For context, a transducer element generally refers to an individual transducer of a transducer array. In many cases, a transducer array includes a set of individual transducers arranged on, for example, a grid of locations. The imaging sub-system 114 can also include a second subset of transducer elements (e.g., transducer elements 114a, 114b, 114c, and 114d) similarly coupled to the subject's skull and configured, during use, to receive ultrasound signals from the portion of the subject's brain in response to the first ultrasound beam. The system 110 includes an electronic controller 112 in communication with ultrasound transducer elements of the imaging sub-system 114 and neuro-modulation sub-system 116 to adjust stimulation based on results from imaging sub-system 114. For example, system 110 can dynamically adjust parameters for emitting ultrasound pulses from the first subset of ultrasound transducer elements based on measured thickness of the subject's skull. In other words, the first subset of ultrasound transducers can direct a second ultrasound beam at the region within the portion of the subject's brain to account for the measured thickness, which can vary within the same subject and from subject to subject.

System 110 provides a high degree of control over stimulation parameters and patterns. System 110 can provide transcranial stimulation by controlling the parameters of pulsed ultrasonic waves to effectuate an ultrasound beam with, for example, a desired focus at a specified region inside the subject's brain. Different stimulation parameters and forms can produce different effects on subject behavior and on the brain. For example, constant stimulation, alternating stimulation, and random noise stimulation can produce different resulting behavior. In some implementations, system 110 can provide transcranial stimulation by driving ultrasound transducers of the neuro-modulation sub-system 116 to emit ultrasound pulses that insonify the subject's brain 104. When the ultrasound pulses converge in a region of the subject's brain (e.g., a cortex), this region may be stimulated and neuro-modulation can be achieved. For example, system 110 can be used to directly stimulate the visual cortex, the auditory cortex, or the somatosensory cortex through ultrasonic stimulation. The methods can also be applied to stimulate peripheral nerves, such as the vagus nerve.

To operate, system 110 can include a wearable headpiece that can be placed on or around a subject's head or neck. In some implementations, system 110 can include a network of individual transducer elements placed on the subject's head or a system that holds individual transducer elements in fixed positions around the subject's head. In this particular example, system 110 can be used without an external power source. For example, system 110 can include an internal power source. The internal power source can be rechargeable and/or replaceable. For example, system 110 can include a replaceable, rechargeable battery pack that provides power to the transducers and sensors.

Subject 102 is a human subject of transcranial stimulation. A focal spot, or target area, within subject's brain 104 can be targeted for intracranial neuro-modulation through skull 162. The target area can be, for example, a specific large-scale brain network associated with a particular state of a subject's brain 104. In some implementations, the target area can be automatically selected based on detection data. For example, the system 110 can adjust the targeted area within subject's brain 104 based on detected brain activity. In some implementations, the target area can be selected manually based on a target reaction from subject's brain 104 or a target reaction from other body parts of the subject. In some implementations, system 110 can stimulate peripheral nerves in addition to brain regions. For example, system 110 can stimulate peripheral nerves such as the vagus nerve to treat affective disorders such as depression or anxiety.

System 110 is shown to include a controller 112, imaging sub-system 114, and neuro-modulation sub-system 116. Imaging sub-system 114 can include transducer elements 114a, 114b, 114c, and 114d, while neuro-modulation sub-system 116 can include transducer elements 116a, 116b, 116c, 116d, 116e, and 116f. The transducer elements can be based on piezo-electric ceramic material, for example, lead zirconate titanate (PZT) materials. The transducer elements can also include capacitive micro-machined transducer (cMUT). In some cases, the imaging sub-system 114 and the neuro-modulation sub-system 116 can use non-overlapping ultrasound transducer elements. In other cases, the imaging sub-system 114 and the neuro-modulation sub-system 116 can have some overlapping ultrasound transducer elements. In both cases, the imaging sub-system 114 may generate one or more images of the subject's brain based on ultrasound signals received in response to transmission events of the neuro-modulation sub-system 116. For example, when ultrasound transducer elements 116a, 116b, 116c, 116d, 116e, and 116f of the neuro-modulation sub-system 116 are activated to transmit ultrasound pulses that insonify a region inside the subject's brain, ultrasound signals may be received at transducer elements 114a, 114b, 114c, and 114d of the imaging sub-system 114. The ultrasound pulses transmitted by ultrasound transducer elements 116a, 116b, 116c, 116d, 116e, and 116f may form a focused ultrasound beam targeting the region of the subject's brain. The focusing can be implemented by judiciously applying an appropriate delay to ultrasound pulses coming off each transducer element so that, once transmitted, the ultrasound pulses can arrive at the targeted region synchronously. Moreover, the amplitude, as well the waveform of the ultrasound pulses, can be adjusted to effectuate, for example, a desired intensity pattern at the targeted region. The skull, including variations of skull thickness, scalp thickness outside the skull, and fatty layer composition inside the skull, can introduce varying propagation delays to the ultrasound pulses, giving rise to a phenomenon known as phase aberration, which can correspond to a blurring, for example, at the intended focus.

Once insonified, ultrasound signals may be reflected from anatomical structures of the subject's brain, including the skull, the scalp, and the region inside the skull. These ultrasound signals may be reflected in response to the ultrasound pulses incident on anatomical structures of the subject's brain. As such, the ultrasound signals may encode anatomic information of the subject's brain. Transducer elements of the imaging sub-system 114 may perform a beamforming reconstruction algorithm to form an image based on the received ultrasound signals. The image may be in the form of a cross-sectional view, rather than a complete tomographic view of the brain. For example, the cross-sectional view may correspond to a portion of the subject's brain and may include sections of the skull for transcranial transmission. The reconstruction algorithm can be a time-of-flight algorithm based on performing delay-and-sum operations to form the image. For example, contributions from the received ultrasound signals can be appropriately delayed and coherently summed to generate an output pixel in the resulting image. Alternatively or additionally, the reconstruction algorithm can include a full-wave inversion (FWI) algorithm or an adaptive wave inversion (AWI) algorithm by solving a non-linear least squares local optimization problem to establish a model of the underlying anatomy that gives rise to the received ultrasound signals in response to the ultrasound pulses transmitted. The FWI, or AWI, approaches can be more computationally time-consuming than time-of-flight algorithms. In some cases, the FWI/AWI approaches can be more sensitive for delineating the underlying anatomy while more likely to be trapped in local optima. In comparison, the time-of-flight algorithm can be less computationally expensive while less prone to local noises.

In more detail, system 110 can use low intensity, pulsed ultrasonic stimulation to stimulate a target area of subject's brain 104. In some implementations, system 110 uses high intensity stimulation subject to thresholds as monitored by system 110 for the subject 102's safety. Here, high intensity refers to intensity levels below the threshold-level to cause irreversible damage, but slightly higher than most extant published studies with human participants. Neuro-modulation sub-system 116 and imaging sub-system 114 can include multiple transducer elements in various arrangements. In example 100 illustrated in FIG. 1A (showing an axial view of the arrangement), transducer elements of the neuro-modulation sub-system 116 can be arranged to closely wrap around the exterior of the skull, for example, on the scalp. In this example, transducer elements (e.g., 114a, 114b, 114c, and 114d) of the imaging sub-system 114 can be arranged slightly off the scalp to interleave with the transducer elements of the neuro-modulation sub-system 116. For one thing, the transducer elements of the imaging sub-system 114 can be smaller than the transducer elements of the neuro-modulation sub-system 116 to provide for less spatial directivity. This reduced directivity can be advantageous when receiving ultrasound signals reflected from the subject's brain. For another, the transducer elements of the imaging sub-system 114 can fill the gap once the transducer elements of the neuro-modulation sub-system 116 are firmly in place (e.g., firming around the scalp and even acoustically matched to the scalp). Here, the transducer elements may be located on the inside of a helmet structure which can provide loose fitting to the subject's head. Once the helmet structure is mounted, adjustments can be made to the locations of the transducer elements of the neuro-modulation system 116 so that these elements closely fit the specific contour of the subject. When necessary, subsequent adjustments can be made to the locations of the imaging sub-system 114 so that these elements, if different from those for neuro-modulation, can be placed at gaps created by transducer elements of the neuro-modulation sub-system 116. In various applications, the subject's hair may be optionally removed for ideal coupling. Notably, the ultrasonic operating frequency of system 110 can be between 200 kHz to 2 MHz (for example, around 500 kHz) where the presence of human hair can be less problematic.

FIG. 1B illustrates example 160, which shows a coronal view of the placement of transducer elements. In this illustration, transducer elements 166a of the neuro-modulation sub-system 116 can be arranged to surround the full circumference of the subject's head. Here, the enclosure can be complete so that the target region of the subject's brain can be insonated from the full range of available angles. In some cases, the full-enclosure arrangement may be preferred to alleviate potential heating at the scalp level when incident acoustic energy is fully distributed to all angles. Here, neuro-modulation sub-system 116 can utilize all combinations of the full aperture to target various areas of the subject's brain. In some cases, multiple areas can be targeted simultaneously to provide efficient coverage of stimulation.

In one illustration, neuro-modulation sub-system 116 can operate according to a Cartesian coordinate system. In this illustration, transducer elements of the neuro-modulation sub-system 116 can be arranged in arrays allow system 110 to dynamically target areas and move the target area in the X, Y, and Z directions. Here, the neuro-modulation sub-system 116 can use phased arrays that can target multiple areas of different depths. The phased arrays allow stimulation generation system 116 to generate and transmit pulsed emissions that have additive effects. In some implementations, neuro-modulation sub-system 116 can include dedicated transducers that target particular beam focal locations. For example, neuro-modulation sub-system 116 can include one or more transducers that are arranged specifically (e.g., curved to match a specific scalp shape, coated with material to acoustically match the impedance of skull) to target a particular area of subject's brain 104.

Neuro-modulation sub-system 116 can include components that enable the system 110 to generate, direct, and focus emissions, including components such as delay lines or zones. For example, stimulation generation system 116 can include delay lines that are arranged specifically for particular transducers and/or particular focal locations within subject 102. In some implementations, multiple neuro-modulation sub-systems may be operated by the system 110 in order to stimulate multiple areas in the brain of subject 102. For example, multiple neuro-modulation sub-systems may be operated in an interleaving fashion to treat multiple foci. The multiple neuro-modulation sub-systems may include multiple types of transducers having different specifications and capabilities. For example, one neuro-modulation sub-system may operate at a higher frequency (e.g., a higher frequency within the range of 200 kHz to 2 MHz) to focus at a target region more close by while another neuro-modulation sub-system can operate at a lower frequency (e.g., a lower frequency within the range of 200 kHz to 2 MHz) to focus on a more distant region (e.g., where ultrasound pulses travel longer distances to converge).

System 110's use of ultrasonic stimulation provides greatly improved spatial precision (e.g., millimeter or sub-millimeter resolution) for localized stimulation as compared to methods that use electrical or magnetic stimulation (e.g., on the order of centimeters). Ultrasound stimulation can target shallow or deep tissue and provides resolution on the order of millimeters. With finer resolution, controller 112 can target deep brain structures such as basal ganglia. For example, controller 112 can use ultrasound stimulation to control tremors by detecting the frequency of a tremor, classifying the frequency as a certain color of noise, and applying stimulation to shift the color of noise.

In various implementations, controller 112 can adjust the intensity, the waveform, as well as the timing of the ultrasound pulses being transmitted to stimulate the subject's brain. In one example, the results from imaging sub-system 114 reveal variation of, for example, the skull thickness over the transmitting ultrasound transducer elements. This means the propagation paths of ultrasound pulses is temporally constant. Based on the variation, controller 112 may adjust the amount of delay for each ultrasound pulse being transmitted from a corresponding ultrasound transducer element to effectively compensate for the altered delays on these propagation paths. Along the same lines, controller 112 may adjust the intensity of the ultrasound pulses being transmitted to compensate for the fact that some propagation paths experience more attenuation over the skull than other propagation paths. In a similar vein, controller 112 may adjust the waveform being transmitted on a given transducer element based on, for example, results from imaging sub-system 114. Here, the results from the imaging sub-system 114 are based on the ultrasound signals received from transducer elements of the imaging sub-system. Examples can include transducer elements 114a, 114b, 114c, and 114d in FIG. 1A, and transducer elements 164a in FIG. 1B. Because the received ultrasound signals travel through the skull as well as the brain, the received signals can be inverted (e.g., by FWI or AWI) to determine skull and scalp variations so that a particular waveform can be selected for transmission that result in a most compact focus, given the propagation paths of ultrasound pulses converging at the focus. Such results may not need to be a full-blown tomographic image of the subject's brain. Rather, results can include an estimate that factors in speed, density, and thickness variations on the propagation paths from a transmitting ultrasound transducer element to a number of receiving ultrasound transducer elements.

System 110 allows contemporaneous or near-contemporaneous detection and stimulation, facilitating a transcranial stimulation system that is able to target large-scale brain networks of subject's brain 104 in real-time and make adjustments to the stimulation based on the detected data. Detection and stimulation may alternate with a period of seconds or less to enable the real-time or near-real-time system. Detection and stimulation signals can be multiplexed. For example, controller 112 can apply stimulation, through the subject's skull, to a target area of subject's brain 104 in-phase with contemporaneous or near-contemporaneous ultrasound imaging, which can operate on ultrasound signals (received by the imaging sub-system 114) in response to stimulating ultrasound pulses (transmitted by the neuro-modulation system 116).

Controller 112 can implement safety measurements to constrain the use of system 110. Controller 112 can monitor the emissions from transducer elements of neuro-modulation sub-system 116 and the subject 102's biological response to the emissions. Controller 112 can receive data from transducer elements on imaging sub-system 114 and other sensing systems communicatively connected to the system 110 and use the data to gauge the stimulation of subject 102 and intensity output of neuro-modulation sub-system 116.

In some implementations, controller 112 can measure the reflection from the skull in response to ultrasonic emissions. Controller 112 can use these reflection measurements to monitor heat levels. For example, controller 112 can use reflection measurements to determine the intensity and timing of the reflections to determine the amount of energy that is currently or cumulatively absorbed by the subject 102. Sustained levels of high intensity emissions can cause injury and/or generate too much heat; controller 112 can adjust stimulation generated by system 110 to control the total thermal dose delivered to the subject 102's scalp or skull.

In some cases, controller 112 monitors the local speed of sound using the ultrasonic pulses emitted. For example, controller 112 can monitor reflections of the ultrasonic emissions from subject 102 to estimate the local speed of sound at the subject 102's body. The speed of sound propagation is dependent on the density of the material from which the sound waves are reflected, and thus is correlated with temperature. This estimation can be used relative to a baseline temperature measurement (e.g., when ultrasound pulses are initially transmitted) for a particular subject 102 and used by controller 112 to monitor, for example, heat levels at the subject 102's skull and head to adjust stimulation. Controller 112 can, for example, determine the local speed of sound at a “cold start,” when stimulation begins, and determine the local speed of sound at a later time, calculating a difference in the amount of time that it takes for the reflected wave to return and thus a change in temperature. Controller 112 can determine, based on a change in the local speed of sound, that the levels of heat being generated from the present stimulation of subject 102 is too high, and can adjust the stimulation by reducing the intensity, stopping the stimulation, etc. for subject 102's safety. For example, controller 112 can continue to monitor the local speed of sound to determine whether to begin stimulation again and/or at what levels the stimulation should be performed.

Controller 112 can also monitor the heat dissipations from subject 102 directly. For example, controller 112 can receive sensor data indicating the subject 102's skin temperature in the scalp area over the targeted brain region and adjust emissions to the subject 102 to limit the intensity of the ultrasound pulses within a safety margin.

Controller 112 can calculate the appropriate phases for therapeutic ultrasound beams that have been steered to the target area of subject's brain 104. These phases can be implemented as timing delays and can interact to increase or decrease resolution and/or power, and can be calculated automatically using various algorithms, including machine learning algorithms as described in more detail below in association with FIG. 2. Controller 112 can automatically determine appropriate phases by changing phases for the ultrasonic output of transducers 116 and use an amount of power returned from the target area to determine whether to change the pressure or phase of each transducer. For example, controller 112 can use the amount of power returned from the target area of subject's brain 104 being stimulated by ultrasonic pulses, and automatically determine a change to the power level of the ultrasound stimulation. Controller 112 can use, for example, phased arrays that emit ultrasound pulses and adjust the phases of these pulses for maximum intensity, up to a predetermined safety threshold level.

System 110 can stimulate target areas of different shapes. For example, system 110 can provide an elongated focus that is not circular. Controller 112 can control transducers 116 to stimulate target areas of different shapes by, for example, steering individual transducers 116 and/or an array of transducers 116. System 110 can stimulate target areas of rectangular, oblong, linear, and triangular shapes among other shapes.

System 110 can identify and target a network of subject's brain 104. For example, system 110 can identify a network of subject's brain 104 to determine multiple target areas to stimulate that will stimulate a target area or produce a desired effect. Controller 112 of system 110 can then stimulate the multiple target areas sequentially or simultaneously to stimulate the target area.

In some implementations, controller 112 can control transducers 116 to stimulate multiple different target areas. For example, controller 112 can focus on or along two distinct points of a particular nerve using a two-dimensional phased array on the neuro-modulation sub-system 116. In some implementations, controller 112 can control transducer elements of multiple arrays of the neuro-modulation sub-system 116 to target one area per array and/or per transducer (so long as the ultrasound pulsed do not interfere). In some implementations, controller 112 controls transducers 116 to simultaneously stimulate two or more target areas. In some implementations, system 110 can stimulate multiple, smaller target areas within a single target area. For example, controller 112 can control transducers 116 to target multiple separate points along a single nerve for additional benefits. Controller 112 can focus multiple transducers 116 on a single target area. For example, controller 112 can control transducers 116 to sync pulses from multiple transducers to match, for example, a measured speed of a pain signal influx.

Controller 112 can control transducers 116 to provide multi-pulse superposition. A pulse at a single focal point makes a pressure wave that propagates radially outward. Controller 112 can use interference effects of ultrasonic emissions to stack a radially propagating pulse with a second pulse at a new position within a target. For example, controller 112 can direct incident ultrasonic pulses to be in phase and at the same frequency to produce a constructive interference. Controller 112 can move the transducers 116 to the new position or steer the transducers 116 to target the new position. Controller 112 can control the steering and focus of the superpositioned ultrasound pulses such that single-pulse thresholds for power are respected while building up displacement with pressure or shear waves from multiple pulses with different focal locations.

Controller 112 can use interference effects of ultrasonic emissions to generate an ultrasonic beat frequency. For example, controller 112 can generate multiple ultrasonic beams with different frequencies to create a beat frequency using both constructive and destructive interference effects. These beat frequencies (related to the differential between the original frequencies) can produce stronger effects than can be achieved using the multiple beams individually. The beat frequencies can, for example, increase spatial resolution and provide non-linear effects. High frequency emissions provide a higher level of precision (by increasing spatial resolution) and low frequency emissions offer a lower level of precision, but travel farther. Controller 112 can use interference effects of ultrasonic emissions, for example, to create a beat envelope that can penetrate the subject 102's skull or other bones around an emission having a frequency that otherwise would not penetrate the subject 102's skull. These applications of the interference effects can give rise to acoustic radiation force imaging (ARFI), as a form of elasticity imaging. In general, controller 112 can operate system 110 to locally stimulate a target area to produce immediate effects, whereas stimulating a particular area such that the acoustic energy transmitted to the area is propagated to a target area can take a longer period of time. Nonetheless, the propagation delay can be a tell-tale sign of local stiffness, which is often times a hallmark feature of elasticity imaging.

Generally, system 110 stimulates subject's brain 104 using ultrasonic stimulation provided by the transducers 116. In some implementations, system 110 can stimulate subject's brain 104 using additional modalities such as electrical or magnetic stimulation. The configuration of system 110's transducers 116 are dependent on the modality of stimulation. For example, in some implementations in which system 110 uses magnetic stimulation techniques, transducers 116 can be located somewhere other than in close proximity to subject 102's head.

System 110 can further include sensors 118 to detect activity of subject's brain 104 by way of electrical, optical, and/or magnetic techniques. For example, system 110 can include non-invasive sensors such as electroencephalogram (EEG) sensors, magnetoencephalograph (MEG) sensors, temperature sensors, infrared sensors, light sensors, heart rate sensors, and blood pressure monitors. Data encoding the detected activity can be stored or made available to controller 112.

Sensors 118 can perform optical detection such that detection does not interfere with the frequencies generated by transducer elements of neuro-modulation sub-system 116. For example, sensors 118 can perform near-infrared spectroscopy (NIRS) or ballistic optical imaging through techniques such as coherence gated imaging, collimation, wavefront propagation, and polarization to determine time of flight of particular photons. Additionally, sensors 118 can collect biometric data associated with subject 102. For example, sensors 118 can detect the heart rate, eye movement, and respiratory rate, among other biometric data of the subject 102. Sensors 118 can provide the collected brain activity data and other data associated with subject 102 to controller 112.

Controller 112 includes one or more computer processors that control the operation of various components of system 110 (including imaging sub-system 114 and neuro-modulation sub-system 116) and components external to system 110. Controller 112 can generate control signals for the system 110 locally. The one or more computer processors of controller 112 can continually and automatically determine control signals for the system 110 without communicating with a remote processing system. For example, controller 112 can receive brain activity feedback data from sensors 118 and results from imaging sub-system 114, both of which can be in response to stimulation from neuro-modulation sub-system 116. Controller 112 may then process the received data and results to determine control signals for neuro-modulation sub-system 116 to fine tune one or more fields generated by neuro-modulation sub-system 116 within the target area of subject's brain 104. In some implementations, controller 112 can also control imaging sub-system 114 to generate, for example, a cross-sectional image of the subject's brain, in response to the subject's brain 104 being stimulated by ultrasound pulses emitted from neuro-modulation system 116. Sensors 118 can also measure brain activity and function through optical, electrical, and magnetic techniques, among other detection techniques. Controller 112 is communicatively connected to sensors 118. In some implementations, controller 112 is connected to sensors 118 through communications buses. In some implementations, controller 112 transmits control signals to components of system 110 wirelessly (e.g., WiFi, Bluetooth, infrared). Controller 112 can receive feedback data from sensors 118, which can be used, along with results from imaging sub-system 114, to adjust subsequent control signals to drive neuro-modulation sub-system 116. In other words, feedback data as well as imaging results in response to stimulation by ultrasound pulses emitted from neuro-modulation sub-system 116 can be used to dynamically adjust subsequent ultrasound pulses to effectuate modified ultrasound stimulation, thereby creating a continuous, closed loop system that is customized for subject 102.

Controller 112 can be communicatively connected to sensors other than sensors 114, such as sensors external to the system 110, and uses the data collected by sensors external to the system 110 in addition to the sensors 114 to generate control signals for the system 110. For example, controller 112 can be communicatively connected to biometric sensors, such as heart rate sensors, eye movement sensors, respiratory rate sensors, and blood pressure monitors that are external to the system 110. In some implementations, the input can include user input. In some implementations, and subject to safety restrictions, a subject can adjust the operation of the system 110 based on the subject's comfort level. Based on the feedback data and/or results from imaging sub-system 114, controller 112 can automatically control system 110 to alter or maintain one or more ultrasound fields generated within the target area of subject's brain 104 by ultrasound pulses emitted from neuro-modulation system 116.

In one illustration, controller 112 uses data collected by sensors 118 and sources separate from system 110 to reconstruct characteristics of brain activity detected in response to stimulation by ultrasound pulses emitted from neuro-modulation subsystem 116, including the location, amplitude, frequency, and phase of large-scale brain activity.

In another illustration, controller 112 can use results from imaging sub-system 114. The results are not limited to a tomographic image of the subject's brain. The results can also include a cross-sectional representation of portions of the subject's brain, such as the skull, the scalp, and some intra-cranial brain tissue. The results can be generated based on received ultrasound signals in response to insonification. In some cases, the insonification may be generated by neuro-modulation sub-system 116. In these cases, imaging sub-system 114 performs image reconstruction in synchronization with the emission of ultrasound pulses from neuro-modulation sub-system 116. In other cases, the insonification is separately provided so that waveforms different from the ones used for neuro-modulation can drive a set of transducer elements. In these cases, the waveforms can have a broader fractional bandwidth than the ones being used for neuro-stimulation. For example, the waveforms for imaging can have a fractional bandwidth of 100%. In comparison, the ultrasound pulses emitted for neuro-stimulation can have a much smaller fractional bandwidth (e.g., 50% or less). A larger fractional bandwidth can be more advantageous to achieve a finer spatial resolution in ultrasound images. A narrower fractional bandwidth, on the other hand, can be more advantageous to pack more acoustic energy to a target region. Additionally, the set of transducer elements can be different from the ones on the neuro-modulation sub-system 116 that were used for neuro-modulation. For example, a tighter focus can be created by a larger aperture involving more ultrasound transducer elements. The arrangement of a larger aperture may be more advantageous for stimulating a finer region of within the subject's brain.

Controller 112 controls the selection of a subset of transducer elements of neuro-modulation sub-system 116 to activate for a particular stimulation pattern. Controller 112 controls the voltage, frequency, and phase for driving the selected subset of transducer elements so that a particular stimulation pattern can be produced. In some implementations, controller 112 uses time multiplexing to create various stimulation patterns using transducer elements of neuro-modulation sub-system 116. In some implementations, controller 112 turns on various combinations of transducer elements on neuro-modulation sub-system 116, which may have differing operational parameters (e.g., voltage, frequency, phase) to create various stimulation patterns.

In some implementations, controller 112 can monitor the subject's use of the system 110 to prevent overuse of the system. For example, controller 112 can monitor levels of use, such as the length of time that the system 110 is used or the strength of the settings at which the system 110 is used, to detect overuse or dependency and perform a safety function such as notifying the subject, stopping the system, or notifying another authorized user such as a healthcare provider. In one example, if the subject uses the system 110 for longer than a threshold period of time that is determined to be safe for the subject, the system 110 can lock itself and prevent further stimulation from being provided. In some implementations, the system 110 can enforce the threshold period of usage for the subject's safety over a period of time, such as 20 minutes of usage within 24 hours. In some implementations, the system 110 can enforce a waiting period between uses, such as remaining locked for 4 hours after a period of usage. Safety parameters such as the threshold period of usage, period of time, and waiting period, among other parameters, can be specified by the subject, the system 110's default settings, a separate system, and/or an authorized user such as a healthcare provider.

Controller 112 can use techniques such as facial recognition, skull shape recognition, among other techniques, for a subject's safety. For example, controller 112 can compare a detected skull shape of a current wearer of the system 110 to determine whether the wearer is an authorized or intended subject. Controller 112 can also select particular models and settings based on the detected subject to personalize stimulation.

Controller 112 allows for input from a user, such as a healthcare provider or a subject, to guide the stimulation. Rather than being fixed to a specific random noise waveform, controller 112 allows a user to feed in waveforms to control the stimulation to a subject's brain.

In some implementations, controller 112 can communicate with a remote server to receive new control signals. For example, controller 112 can transmit feedback from imaging sub-system 114 to the remote server, and the remote server can receive the feedback, process the data, and generate updated control signals for the system 110 and other components.

Controller 112 can perform active, dynamic correction to the stimulation parameters, including the active correction for aberrations in the material through which the ultrasonic emissions will propagate. Such aberrations, such as variations in skull structure, hair, and other materials, can act as a barrier to the ultrasonic emissions and affect the actual impact of the ultrasonic stimulation on subject 102's brain tissue. For example, the skull structure can scatter and/or absorb ultrasonic emissions from system 110 and reduce the impact of the stimulation on subject's brain 104. Controller 112 can dynamically adjust the stimulation parameters to compensate, for example, for variation in skull structure from a baseline model based on sensor data from sensors 114 and data obtained from imaging ultrasonic emissions from transducers 116. In some implementations, controller 112 controls and utilizes lenses and other components to correct for structural aberrations. For example, controller 112 can operate focusing elements such as axicon—a special type of lens that has a conical surface and transforms beams into ring shaped distribution-Fresnel zone plates or Soret—an intense peak in the blue wavelength region of the visible spectrum-zone plates integrated with the transducers. Controller 112 can control elements such as the lenses and/or plates by moving, tilting, applying mechanical stress, applying electro-magnetic fields, and/or applying heat to the elements, among other techniques. In some implementations, each of the one or more transducers 116 includes a custom lens, delay line, or holographic beam former.

Controller 112 can adapt stimulation parameters based on subject 102's bone structure. For example, controller 112 can direct ultrasonic stimulation to different target areas of subject 102 based on the thickness of the bone at that area. In one example, controller 112 can direct stimulation through subject 102's temporal bone window, which is the thinnest part of the skull, in order to stimulate a target area of subject's brain 104 with the minimum amount of skull attenuation. Controller 112 can determine the thickness, shape, size, and/or location, among other characteristics, of particular skeletal structures of subject 102 and use the data to direct stimulation using the structures to aid or amplify the stimulation provided.

System 110 includes safety functions that allow a subject to use the system 110 without the supervision of a medical professional. In some implementations, system 110 can be used by a subject for non-clinical applications in settings other than under the supervision of a medical professional.

In some implementations, system 110 may not be activated by a subject without the supervision of a medical professional, or cannot be activated by a subject at all. For example, system 110 may require credentials from a medical professional prior to use. In some implementations, only subject 102's doctor can turn on system 110 remotely or at their office.

In some implementations, system 110 can uniquely identify a subject 102, and may only be used by the subject 102. For example, system 110 can be locked to particular subjects and may not be turned on or activated by any other users.

System 110 can limit the range of frequencies and intensities of the stimulation applied through transducers 116 to prevent delivery of harmful patterns of stimulation. For example, system 110 can detect and classify stimulation patterns as seizure-inducing, and prevent delivery of seizure inducing stimulus. In some implementations, system 110 can detect activity patterns in early stages of the activity and preventatively take action. For example, system 110 can detect activity patterns in an early stage of anxiety and preventatively take action to prevent subject's brain 104 from progressing into later stages of anxiety. System 110 can also detect seizure activity patterns using the extracranial activity and biometric data collected by sensors 114, and adjust the stimulation provided by transducers 116 to prevent subject 102 from having a seizure. For example, system 110 can be tailored to a subject 102 and used as a brain activity regulation device that detects epileptic activity within the subject's brain 104 and provides prophylactic stimulation.

Controller 112 can build statistical and/or machine learning models based on, for example, ultrasound signals received by transducer elements of imaging sub-system 114 and/or other sensors of system 110. The machine learning models may incorporate a variety of models such as decision trees, linear regression models, logistic regression models, neural networks, classifiers, support vector machines (SVMs), inductive logic programming, ensembles of models (e.g., using techniques such as bagging, boosting, random forests, etc.), genetic algorithms, Bayesian networks. Examples of neural networks can include convolutional neural networks (CNNs) such as U-Net architectures and Residual Networks (Res-Net). The machine learning models may be configured to perform a variety of tasks including, for example, a regression, a classification, a clustering, or a segmentation. The machine learning models can be trained using a variety of approaches, such as deep learning, association rules, inductive logic, clustering, maximum entropy classification, learning classification, etc. In some cases, the machine learning models may use supervised learning. In other cases, the machine learning models use unsupervised learning.

Power system 150 provides power to the various subsystems of system 100 and is connected to each of the subsystems. Power system 150 can also generate power, for example, through renewable methods such as solar or mechanical charging, among other techniques.

In this particular example, power system 150 is shown to be separate from the various other subsystems of system 100. Power system 150 is, in this example, an external power source housed within a separate form factor, such as a waist pack connected to the various subsystems of system 100.

In some implementations, system 100 can be used without an external power source. For example, system 100 can include an integrated power source or an internal power source. The integrated power source can be rechargeable and/or replaceable. For example, system 100 can include a replaceable, rechargeable battery pack that provides power to the transducers and sensors and is housed within the same physical device as system 100.

In this particular example, system 100 is housed within a wearable headpiece that can be placed on a subject's head. In some implementations, system 100 can be implemented as a network of individual transducers and sensors that can be placed on the subject's head or a device that holds individual transducers and sensors in fixed positions around the subject's head. In some implementations, system 100 can be implemented as a device tethered in place and is not portable or wearable. For example, system 100 can be implemented as a device to be used in a specific location within a healthcare provider's office.

Further referring to FIG. 2 showing an example block diagram of a system 200, implementations may incorporate an imaging-driven training engine to optimize transcranial stimulation parameters. In this example, system 200 can analyze results from imaging sub-system 114 to train system 110 for iteratively improved and individualized transcranial stimulation using ultrasound.

As illustrated, system 110 includes a controller 112 that can process results from imaging sub-system 114, and select stimulation parameters for driving neuro-stimulation sub-system 116. As illustrated in FIG. 1 and explained in more detail above, the imaging sub-system 114 includes a set of ultrasound transducer elements to receive ultrasound signals from the subject's brain. The ultrasound signals generally correspond to ultrasound signals reflected from the skull and brain tissues in response to ultrasound insonification by ultrasound pulses the neuro-modulation sub-system 116. The ultrasound signals, once received by the set (or a subset) of ultrasound transducer elements, can be used to generate an estimate as imaging result 214. The estimate can include a tomographic image of the subject's brain. The estimate can also include a cross-sectional image showing a portion of the subject's brain and sections of the skull. The estimate can particularly reveal the variations of skull thickness and scalp thickness. Such results can be used as guidance to further adjustment of stimulation parameters 216. The stimulation parameters may include a waveform pattern, a frequency, a duty cycle, an amplitude or intensity, a temporal delay of the signal driving transducer elements of the neuro-modulation sub-system 116. System 110 may also classify brain activity detected by a sensing system, and use the classification result as feedback data to optimize/adjust stimulation parameters for subsequent neuro-modulation. Classification of brain activities can include identifying the location, amplitude, entropy, frequency, and phase of large-scale brain activity. Controller 112 can additionally perform functions including quantifying dosages and effectiveness of applied neuro-modulation.

In the example of FIG. 2, controller 112 is programmed to pursue data-driven analytics and can include training engine 210 and templates 202. As illustrated, templates 202 are provided to training module 210 as input to train a machine learning model used by controller 112, such as a skull model 212. Templates 202 can serve as seeds to initiate an iterative process to build/develop/refine skull model 212. Templates can include existing data (e.g., ultrasound signals, and/or images reconstructed from ultrasound signals that correspond to known anatomies of brain and skull). These existing data may have been collected under controlled situations when, for example, identical ultrasound pulses were applied to drive an identically selected subset of ultrasound transducer elements for neuro-stimulation of patients. In these controlled situations, the anatomy of brain and skull may have been obtained based on already-established modalities, such as magnetic resonance imaging (MRI) or computer tomography (CT). These controlled situations may also include insonifying, using identical ultrasound pulses emitted from the neuro-modulation sub-system 116, a phantom of a human skull with pre-set skull and scalp parameters, and receiving, using the imaging sub-system 114, ultrasound signals reflected from structures of the phantom. Templates 202 can be derived based on existing ultrasound signals to include an estimate of an existing skull. As noted above, such estimate can include a tomographic image of a known brain, a cross-sectional image showing a portion of the known brain and sections of the known skull. The estimates of existing skulls can be leveraged to infer skull and scalp variations of the next subject.

Based on the imaging result 214 and the templates 202, the training module 210 may iteratively develop skull models 212 to, for example, predict skull and scalp variations for the subject's brain 104. Such skull and scalp variations can be used to adjust stimulation parameters 216 and fine-tune brain stimulation by neuro-modulation sub-system 116. As illustrated above, examples of the skull models 212 can incorporate decision trees, linear regression models, logistic regression models, neural networks (such as convolutional neural networks (CNNs)), classifiers, support vector machines (SVMs), inductive logic programming, ensembles of models (e.g., using techniques such as bagging, boosting, random forests, etc.), genetic algorithms, and Bayesian networks. The skull models 212 may evolve with each addition of imaging result 214 from a subject (or a phantom with known contours and calibrated parameters). In other words, the skull models 212 can be enriched/improved as more imaging results become available. The skull models 212 can be trained through a number of approaches such as deep learning, association rules, inductive logic, clustering, maximum entropy classification, learning classification. The skull models 212 can allow controller 112 to individualize stimulation and treatment to each subject, by using machine learning to select and adjust stimulation parameters for a subject's individual anatomy and brain and/or skull structure.

Controller 112 can access, create, edit, store, and delete models that are tailored to particular common skull structures and/or brain structures. Controller 112 can use different combinations of models for skull structure and brain network structure. Each of these models can be further customized for a subject 102. Controller 112 has access to a set of models that are individualized to a certain extent. For example, controller 112 can use general models for people having a large skull, a small skull, a more circular skull, a more oblong skull, etc. These models provide a starting point that is closer to a subject's skull and brain structures than a single model.

Controller 112 can alter models and create more granularity in the models or otherwise define general models that are often used to be stored within a storage medium available to system 110. Controller 112 can maintain a single model for a particular subject 102 that is improved over time for the subject 102.

As discussed above, system 110 can include sensors 118, which can combine with external sensors to monitor of the effects of neuro-modulation. The monitoring can be performed using various methods of measurement. In some implementations, controller 112 can detect and classify psychological states of a subject's brain 104 based on measured physiological input data including data from electroencephalogram (EEG), magnetoencephalograph (MEG) sensors, as well as measurements of other biometric motion such as eye movement, cardiac or respiratory motion. In some implementations, controller 112 can correlate physiological signals with a subject's brain state. These measured data can be included as feedback data in a closed-feedback stimulation system. In some cases, feedback data can also include user feedback provided through a user interface, such as pushing one button when the effect of stimulation is trending in a positive direction and is achieving a desired effect and pushing a different button when the effect of stimulation is trending in a negative direction and is achieving an undesired effect, among other techniques and modalities of feedback systems.

In some implementations, system 110 can be integrated with a helmet structure that includes a fluid-filled sac or other adjustable, flexible structure that ensures a tight fit on subject 102's head. In some implementations, system 110 can be integrated with a helmet structure that includes an inflatable structure that can be adjusted to exert more or less pressure on subject 102's head to adjust the fit of the helmet.

System 110 can be implemented with a physical form factor that can correct for any aberrations or variations in subject 102's skull structure or other physical features from a general model. For example, system 110 can be implemented as a helmet with a personalized three-dimensional insert. The personalized insert can correct for subject 102's particular variations in skull structure, for example, from a general model of an oval-shaped skull to allow close contact with target portions of subject 102's skull. The personalized insert can be made from material selected for its conductive properties, its texture, etc. In some implementations, controller 112 can control the shape and size of the insert. In some implementations, the insert is fabricated with a fixed shape and can be changed for each subject 102.

In some implementations, the personalized insert can be shaped to provide an improved surface along which transducers are placed and/or through which ultrasonic stimulation is performed. For example, the personalized insert can be shaped to provide a uniform, hemispherical transducer surface. In some implementations, the personalized insert can be shaped to allow all stimulation to arrive at a target area at the same time. The personalized insert can be shaped to provide a reflective surface for the ultrasonic stimulation to direct and/or focus the stimulation. For example, the personalized insert can be shaped to focus the stimulation at a particular target area.

In some implementations, the personalized insert can be shaped to provide a non-uniform surface that is thicker in some areas than in other areas. For example, the personalized insert can be shaped to create a delay line in propagation along a target area. The personalized insert can be shaped based on a calculation of skull thickness performed using imaging techniques as described above or other sensor data collected and provided to controller 112.

In some implementations, the personalized insert can be shaped to create time and/or phase delays in the ultrasonic stimulation. For example, the personalized insert can be shaped to create a phase-delay in ultrasound beams transmitted through the insert based on properties of the material of the insert, including the refractive index, the thickness, and the shape, among other properties. The personalized insert can be designed to correct for anomalous structures and cavities in certain regions of the subject 102's skull by redirecting emissions.

The structure of the personalized insert can be based, for example, on imaging data from, a scan of subject 102's skull that produces a three-dimensional representation of the external structure of the subject 102's skull. For example, the structure of the personalized insert can be determined based on an ultrasound, an MRI, a CT scan or an image of subject 102's skull structure generated from other imaging techniques. In some implementations, the structure of the personalized insert can be based on a general structure of a typical human skull model and adjustments can be made based on imaging data.

Controller 112 can collect response data from subject 102 to quantify dosage provided to subject 102's brain 104. For example, controller 112 can use trained models to quantify dosage based on a response from subject 102's brain 104 to stimulation. System 110 can implement limits on the amount of time that the system 110 can be used, monitor the cumulative dose delivered to various brain areas, enforce a maximum amount of current that can be output by transducers 116, or administer integrated dose control. For example, controller 112 can quantify dosage by measuring physiological responses, such as pupil dilation, to stimulation according to a particular set of parameters. Controller 112 can continuously track eye movement, pupil dilation, and other physiological responses and quantify how effective a particular set of stimulation parameters is. In some implementations, controller 112 can quantify the effectiveness of a particular set of stimulation parameters by monitoring a differential response. For example, controller 112 can effectively “trap and trace” brain signals, such as pain signals, originating from a subject's brain. By comparing the characteristics of the brain signals, controller 112 can detect differential changes in response from a subject 102.

Some implementations can achieve targeting confirmation and ‘dose-response’ quantification using ultrasound for functional brain imaging (e.g., changes in local blood flow) and/or displacement tracking. By combining imaging sub-system 114 and neuro-modulation sub-system 116, controller 112 can track cellular displacement and monitor blood flow concurrently when applying the ultrasound pulses for neuro-modulation. In system 110, imaging can be leveraged for target localization in real time. Brain stiffness/viscoelasticity is an additional biomarker that can be used to track ongoing changes in functional neural activity. In some implementations, imaging sub-system 114 can detect soft tissue changes (e.g., density, temperature, stiffness) by, for example, acoustic radiation force imaging (in the case of transcranial ultrasound stimulation).

During neuro-modulation, imaging sub-system 114 can also monitor stimulation or inhibition of different brain regions by tracking localized changes in blood flow using ultrasound imaging. For context, blood flow and neural activity are known to be intimately linked to each other such that when neural activity increases in a particular brain area, it is accompanied by a localized influx of blood a phenomenon known as functional hyperemia. Functional hyperemia is a rapid response, which onsets with a latency on the order of several seconds, and subsequent return to a baseline level. Some implementations can leverage functional hyperemia as a generally valid proxy measure of local fluctuations in neural activity resulting from the mechanical perturbation of neurons by ultrasound waves. Brain temperature may not be constant, but rather varies continuously with relatively large fluctuations (between 2 to 4 degrees Celsius). Local increases in blood flow and metabolism that are attendant upon greater neural activity are also accompanied by local heat emission. Brain hyperthermia is therefore a normal, physiological and not strictly pathological indicator. In some implementations, imaging sub-system 114 track functional hyperemia at a location inside subject's brain by Doppler flow imaging methodology, such as pulsed Doppler or power Doppler.

In sum, imaging sub-system 114 can monitor, at the focal neuro-modulation spot, local changes in displacement and/or blood flow, as a direct quantitative measure of dosage, i.e., the amount of neuro-modulation delivered to a specific target. By measuring quantitative displacement and/or blood flow, the operator can create a feedback driven closed control loop so that ultrasound transmission can be adjusted on the basis of the quantified dose information.

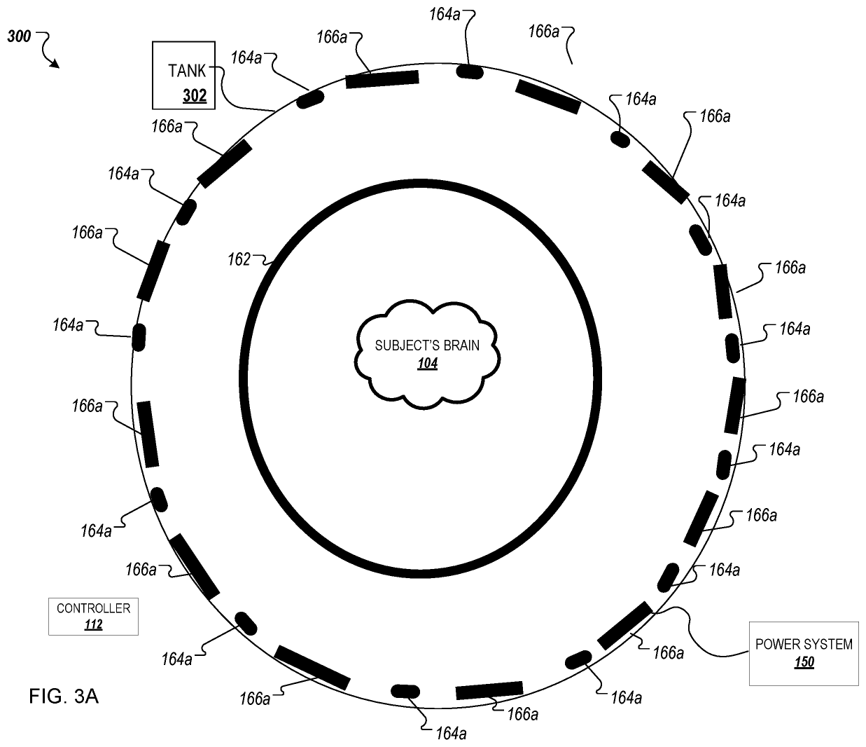

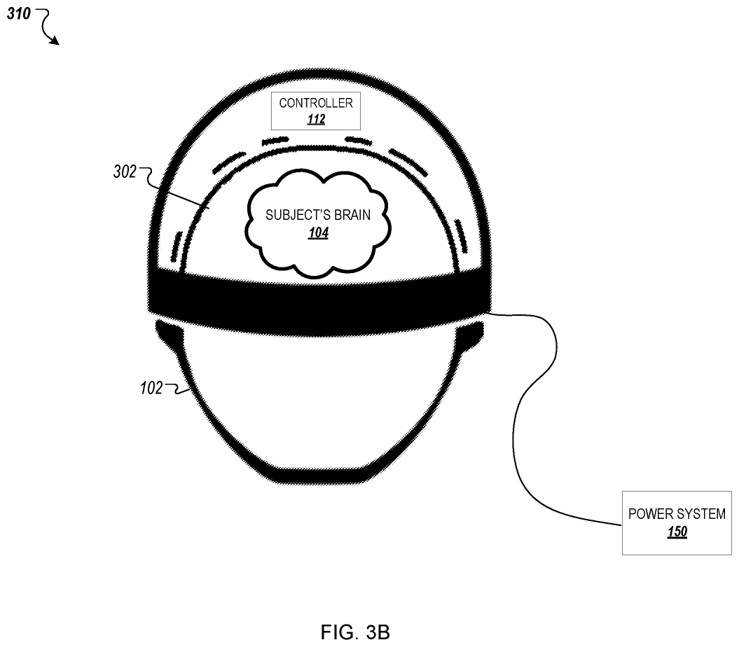

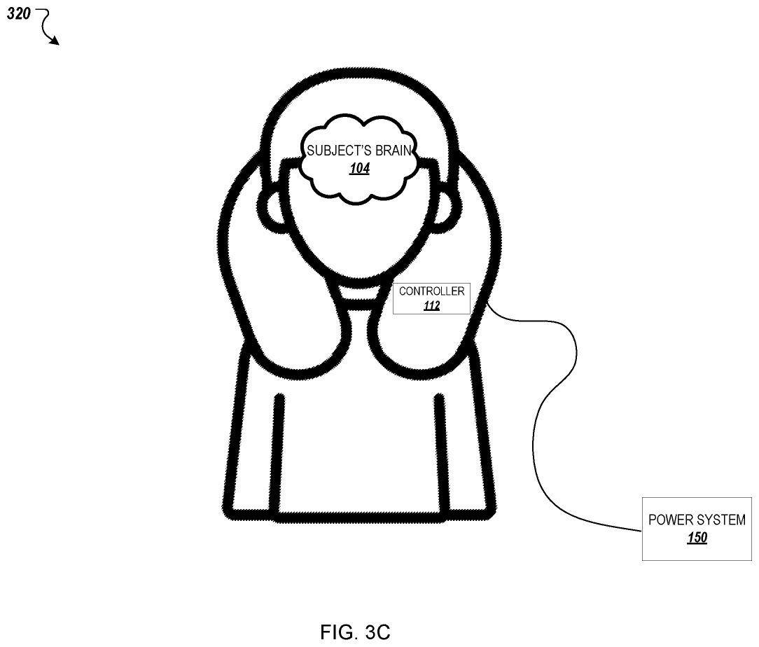

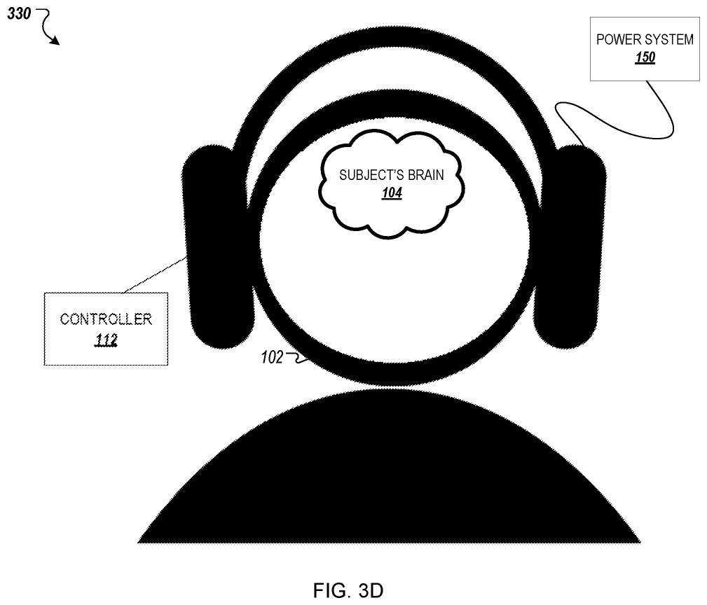

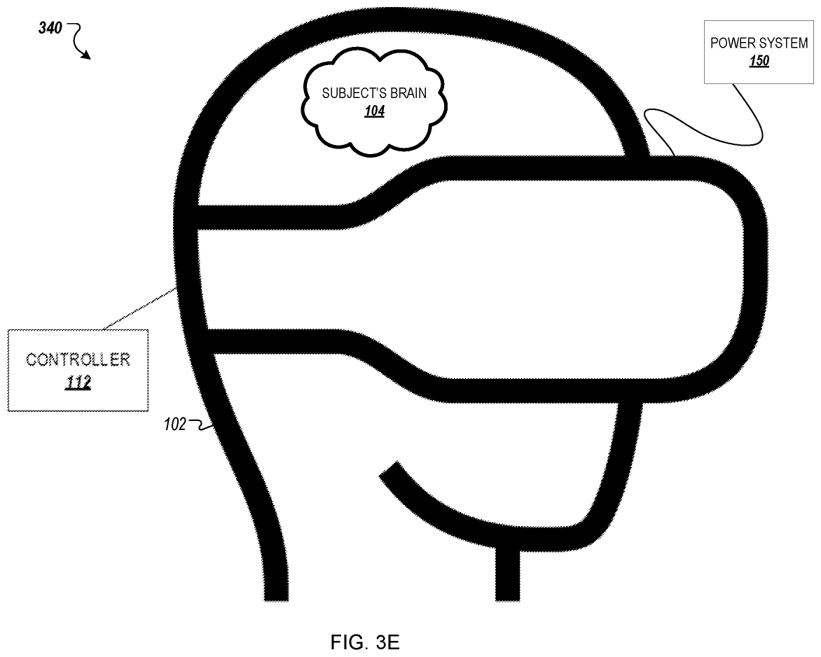

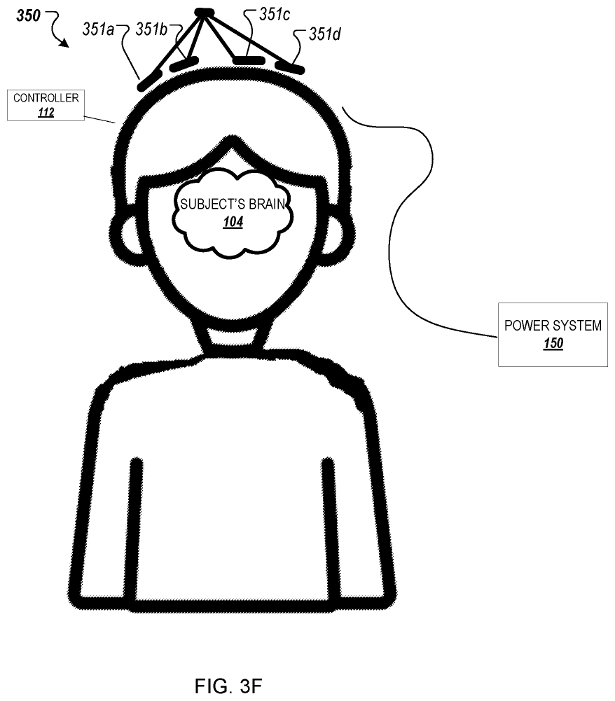

Referring to FIGS. 3A, 3B, 3C, 3D, 3E, and 3F, examples of form factors are presented for a combined transcranial imaging and neuro-modulation system that delivers transcranial stimulation to a target within a subject's brain. For example, system 110 as described above with respect to FIGS. 1A, 1B, and 2 can include form factor devices such as devices 300, 310, 320, 330, 340, or 350 that each includes an imaging sub-system 114 and a neuro-modulation sub-system 116. While controller 112 is depicted as separate from the form factor devices 300, 310, 320, 330, 340, and 350, controller 112 and associated power system 150 can be integrated with the form factor devices of FIGS. 3A, 3B, 3C, 3D, 3E, and 3F to provide a comfortable, more compact packaging. In some implementations, controller 112 communicates with a remote computing device, such as a server, that trains and updates controller 112's machine learning models. For example, controller 112 can be communicatively connected to a cloud-based computing system. As described above, system 110 can include safety features to protect subject 102 and ensure the safe use of system 110. For example, system 110 can include a safety lock-out feature that prevents the transducers 116 from emitting pulses or beamforming if subject 102's head or other body part is not in a correct, safe position relative to the system 110.

When mounting an array of a large number of ultrasound transducer elements for transmitting and receiving operations, the exact relative location of each array element is desired for controlling the transmitting focus and for beamforming to reconstruct the underlying anatomy. To address the challenge, three form factor designs can be applied, namely, a built-in array, a wearable array, and a dynamic array.

An example of a built-in array is represented by device 300 of FIG. 3A. As illustrated, the ultrasound transducer elements (e.g., elements 164a) of the imaging sub-system 114 as well as the ultrasound transducer elements (e.g., elements 166a) of the neuro-modulation sub-system 116 may be mounted inside a water tank (e.g., tank 302). Examples of a water tank can include a salon sink so that a subject may immerse his/her head while keeping the face upward and dry. Each ultrasound transducer is immersed in water and maintains fluidic contact with the subject's skull. The relative locations of each ultrasound transducer element on the inside of the salon sink may then be determined by having the elements pinging each other, like sonar pinging, in an initial calibration procedure. The built-in array approach may accommodate the largest number of transducer elements, among all three approaches.

Examples of a wearable array are represented by FIGS. 3B, 3C, 3D, and 3E. A wearable array may be formed on the inside of these form factor devices. In each example, the wearable array is in a comfortable form factor that contacts subject 102 on multiple points on the head. The wearable array includes multiple transducer elements for imaging sub-system 114 and neuro-modulation sub-system 116 of system 110, as described in FIGS. 1A, 1B, and 2. In each case, the coupling of ultrasound into the skull may be achieved via gel on the tip of each ultrasound transducer element, or refillable fluid in a pouch inside the form factor device. The relative location of each ultrasound transducer element on the inside of the form factor device may be determined by time of flight measurements between each pair of transmit/receive transducer elements. The relative location may also be determined using an external fiduciary marker, or by optical means. Additionally, the helmet-type device may be prescribed based on skull information obtained from the salon-sink approach.

In FIG. 3B, form factor device 310 is a helmet-type device that can be worn by a subject 102 on the head. FIG. 3C illustrates a device 320 that can be worn by a subject 102 around their head and neck. For example, device 320 can take the shape of a pillow that is filled with fluid. The pillow can either be filled fluid for cooling and for coupling ultrasonic waves through the skull to reach the subject's brain. FIG. 3D illustrates a device 330 with the form factor of a pair of headphones that can be worn by a subject on either side of the head. FIG. 3E illustrates a device 340 in the form factor of eyewear that can be worn by a subject 102 on the face. For example, device 340 can resemble a pair of glasses or goggles. In each example, system 110 can be implemented in a flexible, wearable form factor that is portable, wearable, and individualized for a subject 102.

An example of a dynamic array is represented by device 350 of FIG. 3F. As illustrated, device 350 can take the form factor of a scalp scratcher with a group of claws for touching the scalp. A dynamic array of transducer elements (e.g., transducer elements 351a, 351b, 351c, and 351d) can be placed on the tips of the claws of the scalp scratcher. In this configuration, the transducer elements of the dynamic array can be dynamically positioned on the subject's scalp. For example, a suction cup mechanism can be used to achieve firm contact with the subject's scalp. In some cases, the transducer elements can be coated with acoustically matching material. In this arrangement, the relative location of each ultrasound transducer element may be determined by time of flight measurements, using fiduciary markers, or based on optical means.

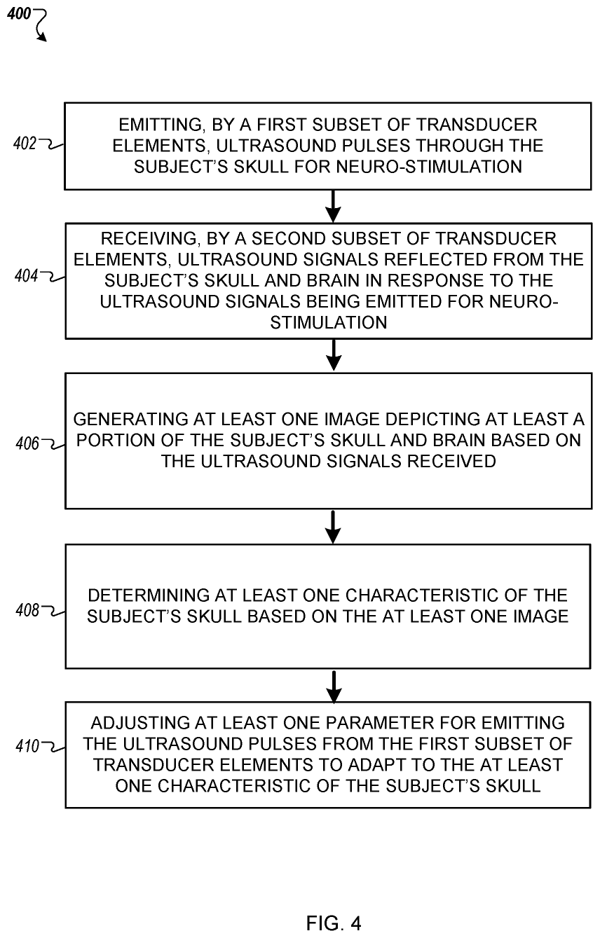

FIG. 4 is a flow chart of an example process 400 of combined transcranial imaging and neuro-stimulation of a subject's brain. Process 400 can be implemented by a combined transcranial imaging and neuro-stimulation system such as system 110 as described above with respect to FIGS. 1, 2, 3A, 3B, 3C, 3D, 3E, and 3F. In this particular example, process 400 is described with respect to system 110 where ultrasound transducer elements of an ultrasound array are mounted on the inside of a portable headset or helmet and coupled to the subject's skull. Briefly, according to an example, the process 400 begins with wrapping the ultrasound transducer elements outside the subject's skull and maintain acoustic contact with the subject's skull.

The process 400 proceeds with emitting, by a first subset of a plurality of transducer elements, ultrasound pulses through the subject's skull for performing a neuro-modulation of the subject's brain (402). As explained above, the first subset of a plurality of transducer elements can correspond to those transducer elements of the neuro-modulation sub-system 116. Once the ultrasound pulses are emitted from the first subset of transducer elements, ultrasound waves can converge in a target region inside the subject's brain 104. For example, controller 112 can apply appropriate delays to each ultrasound pulse being emitted from the first subset of transducer elements so that, after propagation delay, the ultrasound waves can coherently add up in the target area. The controller may additionally control an intensity or amplitude of the ultrasound pulses, a waveform parameter of the ultrasound pulses, a target object, a target size, a target composition, a duration of neuro-modulation, and a particular dosage of neuro-modulation. Examples of waveform parameters can include, a shape of the waveform, a center frequency of the waveform, a bandwidth of the waveform, or a repletion rate for applying the waveform. In some cases, the bandwidth of the waveform can be expressed as a fractional bandwidth, e.g., the percentage of the ratio of the absolute bandwidth (e.g., in Hz) over the center frequency. In some cases, the transducer elements can operate at a center frequency between 200 kHz and 2 MHz (e.g., around 500 Hz). In some cases, the transducer elements can have a fractional bandwidth as large as 100%.

The process 400 continues with receiving, by a second subset of a plurality of transducer elements, ultrasound signals reflected from the subject's brain and skull in response to the ultrasound pulses being emitted from the first subset of the plurality of transducer elements (404). As explained above, the second subset of a plurality of transducer elements can correspond to those transducer elements of the imaging sub-system 114. For example, controller 112 can operate transducer elements of the imaging sub-system 114 so that each transducer element receives an instance of an ultrasound signal as reflected from the subject's brain and skull. The first and second subsets of transducer elements can overlap. In other words, one or more transducer elements can be configured in transceiver mode. Each of the second subset of transducers can be smaller in size (e.g., smaller area, smaller width, smaller height) than at least one transducer element from neuro-modulation sub-system 116. The first and the second subset of the plurality of transducer elements are tuned to operate between 200 kHz to 2 MHz (e.g., around 500 kHz) and the transducer elements on the imaging sub-system 114 can have a broader bandwidth for more sensitive detection/reception.

The process 400 continues with generating at least one image depicting at least a portion of the subject's skull and brain based on, at least in part, the ultrasound signals received by the second subset of the plurality of transducer elements (406). The at least one image can include a tomographic image of the subject's brain. The at least one image can also include a cross-sectional image depicting a portion, rather than a full tomographic view, of the subject's brain. As explained above, the at least one image can be derived from the instances of the ultrasound signals received by the transducer elements of the imaging sub-system 112. In some cases, a time-of-flight algorithm based on performing delay-and-sum operations can generate the at least one image. For example, the instances of the received ultrasound signals can be appropriately delayed and coherently summed to generate an output pixel in the resulting image. In other cases, a full-wave inversion (FWI) algorithm or an adaptive wave inversion (AWI) algorithm can generate at least one image by solving a non-linear least squares local optimization problem to establish a model of the underlying anatomy.

Based on, at least in part, the at least one image, process 400 can adapt the neuro-modulation of the subject's brain. For example, the process 400 can determine at least one characteristic of the subject's skull based on, at least in part, the at least one image (408). The at least one characteristic can include skull thickness and scalp thickness that are specific to the subject. The at least one characteristic can also include variations of skull thickness and scalp thickness over a surface area of the subject's skull where the ultrasound pulses are launched from the transducer elements of the neuro-modulation sub-system 116. The process 400 may then adjust at least one parameter for emitting the ultrasound pulses from the transducer elements of the neuro-modulation sub-system 116 to adapt to the at least one characteristic of the subject's skull (410). For example, controller 112 may adjust a delay parameter for each ultrasound pulse being emitted from a corresponding ultrasound transducer element of the neuro-modulation sub-system 116 to effectively compensate for the altered delays on these propagation paths. Controller 112 may adjust an intensity parameter of the ultrasound pulses being emitted to compensate for the fact that some propagation paths experience more attenuation over the skull than other propagation paths. In a similar vein, controller 112 may adjust a waveform shape of the ultrasound pulse being emitted from a given transducer element.

Process 400 may be implemented as a feedback controlled loop when the adjusted emission parameter (410) is applied to subsequent emissions of ultrasound pulses for neuro-modulation (402). Process 400 may incorporate training skull models based on results from the imaging sub-system 114. As explained above, controller 112 can access a plurality of templates that comprise data encoding images of known skulls. These images may have been generated based on ultrasound signals received from the known skulls in response to the ultrasound pulses being directed to the known skulls from the first subset of the plurality of transducer elements. The templates represent known knowledge where a set of parameters for applying neuro-modulation is optimized for a particular skull. These templates may be stored in a memory component of the controller 112. Controller 112 may be further configured to operate a training engine 210. As explained above, training engine 210 can operate on the templates and the at least one image (specific to the subject) to infer a desirable set of pulse parameters for adapting the neuro-modulation to the subject. For example, training engine 210 can build skull models with each addition of image and selected set of pulse parameters. Each skull model comprises at least one feature that corresponds to a skull thickness.

A number of implementations have been described. Nevertheless, it will be understood that various modifications may be made without departing from the spirit and scope of the disclosure. For example, various forms of the flows shown above may be used, with steps re-ordered, added, or removed.

All of the functional operations described in this specification may be implemented in digital electronic circuitry, or in computer software, firmware, or hardware, including the structures disclosed in this specification and their structural equivalents, or in combinations of one or more of them. The techniques disclosed may be implemented as one or more computer program products, i.e., one or more modules of computer program instructions encoded on a computer-readable medium for execution by, or to control the operation of, data processing apparatus. The computer readable-medium may be a machine-readable storage device, a machine-readable storage substrate, a memory device, a composition of matter affecting a machine-readable propagated signal, or a combination of one or more of them. The computer-readable medium may be a non-transitory computer-readable medium. The term “data processing apparatus” encompasses all apparatus, devices, and machines for processing data, including by way of example a programmable processor, a computer, or multiple processors or computers. The apparatus may include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) may be written in any form of programming language, including compiled or interpreted languages, and it may be deployed in any form, including as a standalone program or as a module, component, subroutine, or other unit suitable for use in a computing environment. A computer program does not necessarily correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform functions by operating on input data and generating output. The processes and logic flows may also be performed by, and apparatus may also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory may be supplemented by, or incorporated in, special purpose logic circuitry.

To provide for interaction with a user, the techniques disclosed may be implemented on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input.

Implementations may include a computing system that includes a back end component, e.g., as a data server, or that includes a middleware component, e.g., an application server, or that includes a front end component, e.g., a client computer having a graphical user interface or a Web browser through which a user may interact with an implementation of the techniques disclosed, or any combination of one or more such back end, middleware, or front end components. The components of the system may be interconnected by any form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

Thus, particular implementations have been described. Other implementations are within the scope of the following claims. For example, the actions recited in the claims may be performed in a different order and still achieve desirable results.

What is claimed is:

1. A system for imaging and neuro-modulation of a subject's brain through the subject's skull, the system comprising:
an ultrasound array comprising a plurality of transducer elements, wherein:
a first subset of the plurality of transducer elements are configured to emit ultrasound pulses through the subject's skull for performing a neuro-modulation of the subject's brain during use of the system, and
a second subset of the plurality of transducer elements are configured to receive ultrasound signals from the subject's skull and brain in response to the ultrasound pulses being emitted from the first subset of the plurality of transducer elements; and
a controller coupled to the ultrasound array, wherein the controller is configured, during use of the system, to:
generate at least one image depicting at least a portion of the subject's skull and brain based on, at least in part, the ultrasound signals received by the second subset of the plurality of transducer elements, and
adapt the neuro-modulation of the subject's brain based on, at least in part, the at least one image.

2. The system of claim 1, wherein the controller is further configured, during use of the system, to:
determine at least one characteristic of the subject's skull based on, at least in part, the at least one image, and
adjust at least one parameter for emitting the ultrasound pulses from the first subset of the plurality of transducer elements to adapt to the at least one characteristic of the subject's skull.

3. The system of claim 2, wherein the controller is further configured, during use of the system, to store a plurality of templates,
wherein the plurality of templates comprise data encoding images of known skulls, and
wherein the images are generated based on ultrasound signals received from the known skulls in response to the ultrasound pulses being directed to the known skulls from the first subset of the plurality of transducer elements.

4. The system of claim 3, wherein the controller is further configured, during use of the system, to train skull models based on, at least in part, the plurality of templates and the at least one image specific to the subject; and
wherein each skull model comprises at least one feature that corresponds to a skull thickness.

5. The system of claim 4, wherein when the at least one parameter is adjusted, the ultrasound pulses being emitted from the first subset of the plurality of transducer elements are adapted to the skull thickness specific to the subject.

6. The system of claim 1, wherein the first subset of the plurality of transducer elements and the second subset of the plurality of transducer elements share a common group of transducer elements.

7. The system of claim 1, wherein each of the second subset of the plurality of transducer elements is sized to be smaller than at least one transducer element from the first subset of the plurality of transducer elements.

8. The system of claim 1, wherein the first and the second subset of the plurality of transducer elements are tuned to operate between 200 kHz to 2 MHz.

9. The system of claim 1, wherein the at least one image comprises a tomographic image of the subject's brain.

10. The system of claim 1, wherein the first subset of the plurality of transducer elements are configured, during use of the system, to surround the subject's skull.

11. A method for imaging and neuro-modulation of a subject's brain through the subject's skull, the method comprising:
emitting, by a first subset of a plurality of transducer elements, ultrasound pulses through the subject's skull for performing a neuro-modulation of the subject's brain;
receiving, by a second subset of the plurality of transducer elements, ultrasound signals reflected from the subject's brain and skull in response to the ultrasound pulses being emitted from the first subset of the plurality of transducer elements;
generating at least one image depicting at least a portion of the subject's skull and brain based on, at least in part, the ultrasound signals received by the second subset of the plurality of transducer elements; and
adapting the neuro-modulation of the subject's brain based on, at least in part, the at least one image.

12. The method of claim 11, further comprising:
determining at least one characteristic of the subject's skull based on, at least in part, the at least one image, and
adjusting at least one parameter for emitting the ultrasound pulses from the first subset of the plurality of transducer elements to adapt to the at least one characteristic of the subject's skull.

13. The method of claim 12, further comprising:
accessing a plurality of templates that comprise data encoding images of known skulls, wherein the images are generated based on ultrasound signals received from the known skulls in response to the ultrasound pulses being directed to the known skulls from the first subset of the plurality of transducer elements.

14. The method of claim 13, further comprising:
building skull models based on, at least in part, the plurality of templates and the at least one image specific to the subject, wherein each skull model comprises at least one feature that corresponds to a skull thickness.

15. The method of claim 14, wherein when the at least one parameter is adjusted, the ultrasound pulses being emitted from the first subset of the plurality of transducer elements are adapted to the skull thickness specific to the subject.

16. The method of claim 11, wherein the first subset of the plurality of transducer elements and the second subset of the plurality of transducer elements share a common group of transducer elements.

17. The method of claim 11, wherein each of the second subset of the plurality of transducer elements is sized to be smaller than at least one transducer element from the first subset of the plurality of transducer elements.

18. The method of claim 11, wherein the first and the second subset of the plurality of transducer elements are tuned to operate between 200 kHz to 2 MHz.

19. The method of claim 11, wherein the at least one image comprises a tomographic image of the subject's brain.

20. The method of claim 11, further comprising: arranging the first subset of the plurality of transducer elements to surround the subject's skull.
